(19) United States
(12) Patent Application Publication
(10) Pub. No.: US 2006/0206037 A1
(43) Pub. Date: Sep. 14, 2006

(54) Apparatus and method for non-invasive measurement of intracranial pressure

(76) Inventor: Ernest E. Braxton, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 11/362,364

(22) Filed: Feb. 24, 2006

Related U.S. Application Data
(60) Provisional application No. 60/656,449, filed on Feb. 24, 2005.
(60) Provisional application No. 60/703,391, filed on Jul. 29, 2005.

(51) Int. Cl. A61B 5/00; A61B 5/04

(52) U.S. Cl. 600/561; 600/395

(57) ABSTRACT

An intracranial pressure (ICP) within a patient's skull can be determined by observing a vessel in the patient's eye, desirably in the red and/or infrared (IR) spectrum, while causing the pressure inside the eye to increase. On or about the time the observed vessel collapses in response to increasing the pressure inside the eye, the pressure inside the eye is determined. The ICP can then be determined as a function of the pressure inside the eye. Desirably, the vessel being observed is the central retinal vein of the eye.

### CROSS REFERENCE TO RELATED APPLICATIONS

The present application claims priority from U.S. Provisional Application No. 60/656,449, filed Feb. 24, 2005, and U.S. Provisional Patent Application No. 60/703,391, filed Jul. 29, 2005, both of which are incorporated herein by reference.

### BACKGROUND OF THE INVENTION

1. Field of the Invention

The present invention relates to determining intracranial pressure in patients and, more particularly, to determining intracranial pressure by non-invasively determining the point when one or more vessels with the eye of a patient collapses when a known load is applied to the exterior of the eye.

2. Description of Related Art

Intracranial pressure (ICP) is an important parameter in the management of conditions such as traumatic brain injury, stroke, intracranial hemorrhage, central nervous system (CNS) neoplasm, CNS infections and hydrocephalus where cerebral edema exists or brain compliance is altered. High ICP must be aggressively treated to prevent secondary neurological damage. ICP may vary widely when cerebral edema exists; therefore continuous or semi-continuous measurement of ICP is very useful to gauge the effectiveness of treatment.

Heretofore, most current methods for measuring ICP involve the intracranial surgical placement of a fluid coupled strain gauge or fiber-optic pressure transducer. These apparatus and the surgical procedures required for their invasive insertion have many untoward side effects such as bleeding, infection, malfunction, and herniation that may result in permanent disability or death.

Other proposed non-invasive methods and apparatus to measure ICP have not been adapted to medical practice due to practical limitations preventing their use in real world clinical practice. Such proposed techniques include measuring evoked otoacoustic emissions, ultrasonic detection of the optic nerve or vessels, pulse phase-locked loop ultrasonation of the cranium, transcranial doppler (TCD) ultrasonography of the cerebral arteries, dynamic magnetic resonance imaging (dMRI), optical coherance tomography (OCT) of the optic nerve sheath, (ONS) and manual ophthalmodynamometry using a traditional direct or indirect fundoscopy.

As reported by Buki et al. in Hearing Research 94 (1996) pp. 125-139, evoked otoacoustic emissions can, in theory, measure ICP through communication between the cerebrospinal fluid (CSF) space and the perilymphatic fluid of the scala tympanani. However, this method is limited both by the fact that a significant percent of the normal population lack this CSF communication due to a normal anatomical variation and by the indirect nature of otoacoustic emission measurement.

A proposed non-invasive method of ICP measurement through pulse phase-locked loop ultrasonation of the cranium is disclosed in U.S. Pat. No. 6,475,147 to Yost et al. In the Yost et al. patent, ICP is deduced by correlating changes in the pulsatile components of the CSF. This technique is encumbered by the clinically complicated calibration process of tilting the patient's head which can be contraindicated in trauma patients with suspected cervical and spinal injuries. This method also requires an intact skull, making it impractical for patients who have skull fractures or surgical opening of the skull during brain surgery.

A non-invasive method of ICP measurement through the transcranial doppler (TCD) ultrasonography of the cerebral arteries has also been proposed. However, this technique has limited practical use because of the unpredictable nature of the brain's cerebrovascular autoregulatory mechanisms.

Correlation of ICP to the optic nerve sheath thickness with OCT or ultrasound is described in U.S. Pat. No. 6,129,682 to Borchert et al. The relevance of the measurement disclosed in the Borchert et al. patent is doubtful because onset of papilledema may be delayed 2 to 4 hours after the onset of high ICP. This deficiency is clinically significant because as ICP increases, cerebral perfusion decreases, leading to decreased brain oxygenation and metabolism. This 2-4 hour delay can lead to preventable brain injury or even death. Additionally, a significant percentage of patients with documented ICP elevation lack the ostensible changes in the optic nerve that the technique disclosed in the Borchert et al. patent seeks to identify.

Current methods of ophthalmodynamometry using traditional direct or indirect fundoscopy require a high level of technical training to successfully perform and are subject to inter-observer variability. An example of an ophthalmodynamometry technique using a hand-held direct ophthalmoscope can be found in U.S. Patent Application Publication No. 2004/0230124 to Querfurth.

Other prior art related to determining ICP includes:
U.S. Pat. No. 4,907,595 to Strauss;
U.S. Pat. No. 5,951,477 to Ragauskas et al.;
U.S. Pat. No. 6,027,454 to Löw;
B. BÜKI, P. AVAN, J.J. LEMAIRE, M. DORDAIN, J. CHAZAL and O. RIBÁRI; “Otoacoustic Emissions: A New Tool For Monitoring Intracranial Pressure Changes Through Stapes Displacements”; Hearing Research 94 (1996), pp. 125-139;
M. MOTSCHMANN, C. MÜLLER, M. SCHÜTZE, R. FIRSCHING and W. BEHRENS-BAUMANN; “Ophthalmodynamometry—A Reliable Method For Non-Invasive Measurement Of Intracranial Pressure”; http://www.dog.org/1999/e-abstract99/678.html, 2 pages;
DRAEGER J, RUMBERGER E, and HECLER B.; “Intracranial Pressure In Microgravity Conditions: Non-Invasive Assessment By Ophthalmodynamometry”; Aviat Space Environ Med. 1999 December; 70(12): pp. 1227-79;
RAIMUND FIRSCHING, M.D., MICHAEL SCHÜTZE, M.D., MARKUS MOTSCHMANN, M.D., and WOLFGANG BEHRENS-BAUMANN, M.D.; “Venous Ophthalmodynamometry: A Noninvasive Method For Assessment Of Intracranial Pressure”; J. Neurosurg./Volume 93/July, 2000; pps. 33-36;
MOTSCHMANN M, MÜLLER C, WALTER S, SCHMITZ K, SCHÜTZE M, FIRSCHING R and BEHRENS-BAUMANN W; “Ophthalmodynamometry. A Reliable Procedure For Noninvasive Determination Of Intracranial Pressure”; Ophthalmologe. 2000 December; 97(12): pp 860-62;
MOTSCHMANN M, MÜLLER C, KUCHENBECKER J, WALTER S, SCHMITZ K, SCHÜTZE M, FIRSCHING R and BEHRENS-BAUMANN W and FIRSCHING R; “Ophthalmodynamometry. A Reliable Method For Measuring Intracranial Pressure”; Strabismus. 2001 March; 9(1): pp. 13-6; and
MEYER-SCHWICKERATH R, STODTMEISTER R, and HARTMANN K.; “Non-Invasive Determination Of Intracranial Pressure. Physiological Basis And Practical Procedure”; Kiln Monatsbl Augenheikd. 2004 December; 221(12):pp 1007-11.

### SUMMARY OF THE INVENTION

The present invention is a method of non-invasively determining an intracranial pressure (ICP) of a patient. The method includes (a) observing a vessel in a patient's eye; (b) causing a pressure inside the eye to increase; (c) determining when the observed vessel collapses in response to increasing the pressure inside the eye; (d) estimating the pressure inside the eye on or about the time the vessel collapses; and (e) estimating the ICP as a function of the estimated pressure inside the eye.

As used herein, the term non-invasively means not entering or penetrating the body.

The method can further include determining a resting pressure inside the eye and estimating the ICP as a function of the combination of the resting pressure and the estimated pressure inside the eye.

Step (a) can include causing light to shine into the patient's eye; electronically acquiring a plurality of images from the patient's eye when the light is shining therein; and electronically processing each acquired image. The light can be light in the red and/or infrared (IR) spectrum and/or each image can be acquired in the red and/or IR spectrum.

Step (c) can include electronically determining when the vessel collapses automatically from the plurality of electronically processed images. The vessel can be observed at wavelengths between 400-2500 nm, desirably between 400-1000 nm, more desirably between 500-1000 nm, even more desirably between 600-1000 nm and most desirably between 600-700 nm.

Step (a) can be accomplished by detecting blood volume in the vessel in the red and/or IR spectrum. Step (c) can be accomplished by detecting a reduction in the blood volume in at least a portion of the vessel in the red and/or IR spectrum.

Desirably, the vessel is the central retinal vein of the eye.

The invention is also an apparatus for non-invasively determining an intracranial pressure (ICP) of a patient. The apparatus includes a camera for electronically acquiring a plurality of images of an interior of an eye of the patient; a pressure loading device for non-invasively applying a load to an exterior of the eye to increase a pressure inside the eye; a load detector for electronically determining the load applied to the exterior of the eye by the pressure loading device; and a controller for processing the images acquired by the camera to automatically determine when a vessel in the interior of the eye collapses in response to increasing the pressure inside the eye, for acquiring from the load detector the load applied to the exterior of the eye on or about the time the vessel collapses, and for determining the ICP as a function of the load applied to the exterior of the eye on or about the time the vessel collapses.

A light source can shine light into the interior of the eye. The light can be light in the red and/or infrared (IR) spectrum. The camera can be configured to acquire images in the red and/or IR spectrum.

The apparatus can further include a system for determining a resting pressure inside the eye in the absence of a load being applied to the exterior of the eye. The controller can determine the ICP as a function of the combination of resting pressure inside the eye and the load applied to the exterior of the eye on or about the time the vessel collapses.

The controller can automatically determine when the vessel collapses by comparing two or more of the acquired images and determining from said comparison when a reduction in the amount of blood volume in the vessel occurs.

Lastly, the invention is a method of non-invasively determining an intracranial pressure (ICP) of a patient. The method includes (a) acquiring electronic images of a vessel in an interior of a patient's eye; (b) applying an increasing load to the exterior of the eye, whereupon a pressure inside the eye increases, until the vessel is determined to collapse from the acquired images; (c) determining the load applied to the exterior of the eye on or about the time the vessel collapses; and (d) estimating the ICP as a function of the load applied to the exterior of the eye on or about the time the vessel collapses.

Desirably the electronic images are acquired in the red and/or infrared (IR) spectrum.

The method can include converting the red and/or IR electronic images into corresponding images in the visible spectrum and manually determining when the vessel collapses from the images in the visible spectrum. Alternatively, the method can include automatically determining when the vessel collapses from the acquired electronic images; automatically determining the load applied in step (c); and automatically determining the ICP in step (d).

The method can further include determining a resting pressure inside the eye, in a manner known in the art, in the absence of a load being applied to the exterior of the eye. Step (d) can include estimating the ICP as a function of the resting pressure.

Step (c) can include estimating an actual load applied to the eye based on the load determined to be applied to the exterior of the eye on or about the time the vessel collapses and based on at least one characteristic of the device used to apply the increasing load to the eye. Step (d) can include summing the estimated actual load applied to the eye and the resting pressure.

The means used to apply the increasing load to the eye can include a means for applying either a negative pressure (vacuum) or a positive pressure (pressing force) to the exterior of the eye. The means for applying the negative pressure can include a suction cup coupled to a source of vacuum. One of the characteristics used to estimate the actual load applied to the eye can include the diameter of the suction cup. The load determined to be applied to the exterior of the eye on or about the time the vessel collapses can be determined from a measurement of the vacuum applied to the suction cup.

### BRIEF DESCRIPTION OF THE DRAWINGS

FIG. 1 is a combined schematic and diagrammatic view of an apparatus in accordance with the present invention positioned relative to an eye of a patient for determining the intracranial pressure (ICP) of the patient; and

FIG. 2 is a flow diagram of a method for determining ICP.

### DETAILED DESCRIPTION OF THE INVENTION

The present invention will be described with reference to the accompanying figures.

Within a human eye, the optic nerve travels through the cerebral spinal fluid (CSF) space before entering the interior of the eye. There are two major vessels that run in the optic nerve sheath, namely, a high-pressure central retinal artery and a low-pressure central retinal vein. Other vessels, such as arterioles, capillaries and venuoles, are tributaries of the central retinal artery and the central retinal vein in the eye. The pressure in the central retinal vein (CRV) must be greater than the intracranial pressure (ICP) surrounding the optic nerve sheath in order for blood to flow through the optic nerve sheath. The pressure required to collapse the CRV, called the venous outflow pressure (VOP), may be used to determine ICP.

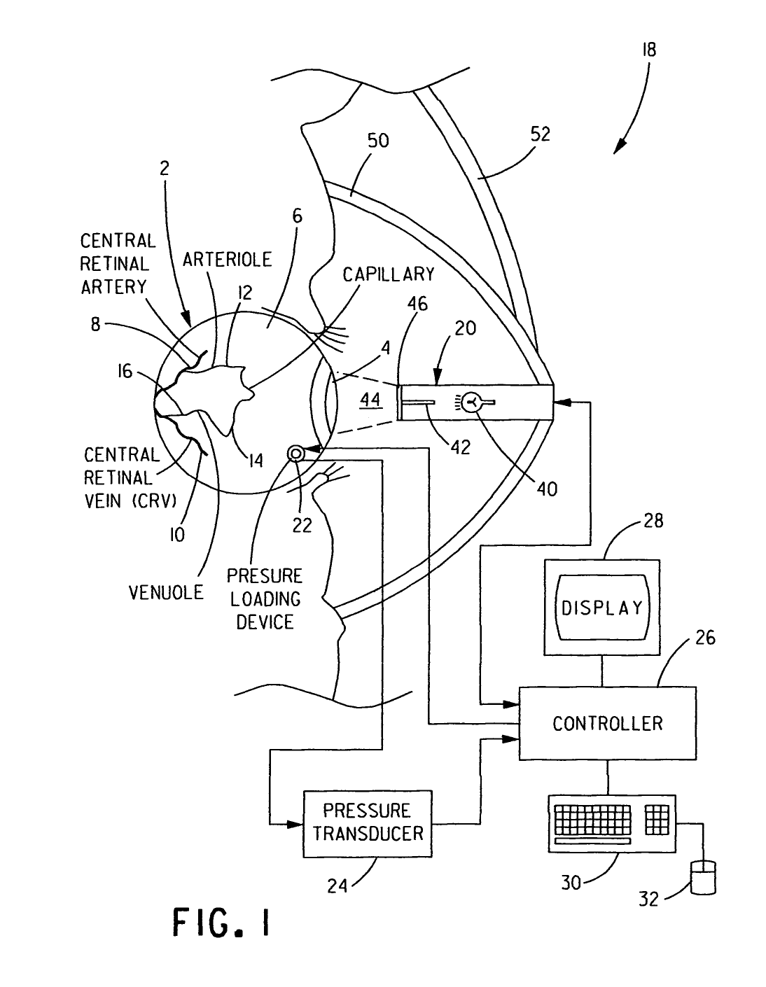

With reference to FIG. 1, a human eye 2 includes a cornea 4 and a sclera 6. An interior of eye 2 includes a central retinal artery 8, a central retinal vein 10, one or more arterioles 12, one or more capillaries 14 and one or more venuoles 16.

An apparatus 18 for non-invasively measuring ICP includes an imaging device 20, a pressure loading device 22, a pressure transducer 24 and a controller 26. A human machine interface (HMI), comprising a display 28, a keyboard 30 and a mouse 32, can be coupled to controller 26 to facilitate interaction between controller 26 and an attendant (not shown).

Imaging device 20 can include or have associated therewith an illumination device 40, such as, without limitation, a lamp, the combination of an optical fiber and a lamp, and the like, for illuminating the interior of eye 2 and a camera 42 which converts optical images acquired of the interior of eye 2 in the field-of-view 44 of camera 42 into analog or digital signals for processing by controller 26 in a manner to be described hereinafter. For the purpose of describing the present invention, illumination device 40 is illustrated as a lamp inside a housing of imaging device 20. However, this is not to be construed as limiting the invention since it is envisioned that illumination device 40 can be any suitable and/or desirable device for illuminating the interior of eye 2 and said device can reside in any suitable and/or desirable location inside or outside of the housing of imaging device 20.

Light output by illumination device 40 is directed into the interior of eye 2, desirably via cornea 4. Light from illumination device 40 entering eye 2 is reflected by internal structure of eye 2 such as, without limitation, CRV 10, to form the optical images acquired by camera 42 from field-of-view 44.

Desirably, the light entering eye 2 and/or the light detected by camera 42 is light having wavelengths between 400-2500 nm, desirably between 400-1000 nm, more desirably between 500-1000 nm, even more desirably between 600-1000 nm and most desirably between 600-700 nm. For reasons discussed next, light in the red and/or infrared (IR) spectrum is particularly desirable for illuminating the interior of eye 2.

Red and/or IR light is particularly useful for non-invasively measuring ICP in accordance with the present invention for a number of reasons. First, the use of red and/or IR light of suitable wavelenght(s) allows identification of the central retinal artery from the central retinal vein based on the different light refactory characteristics of oxygenated blood in the artery and deoxygenated blood in the vein. Second, red and/or IR light enables improved accuracy and precision in determining the collapse of the preferred vessel, i.e., CRV 10, for ICP correlation. This is because the size of CRV 10 can be further distinguished based on its optical properties. A third benefit of utilizing red and/or IR light is that it allows imaging of the retinal vessels in light spectrums not visible to the human eye thereby avoiding the potentially harmful effects thereof on the eye. Imaging in non-visible wavelengths also allows the patient's pupil to dilate without the use of pharmacological agents. This offers distinct advantages in clinical evaluation of neurological patients as the unaltered size of a patient's pupil is a critical part of a neurological exam. The pharmacological dilation of the pupil artificially dilates the pupil for a prolonged period, interfering with this important portion of serial clinical neurological examinations. Lastly, red and/or IR radiation enables camera 42 to view the vessels of the eye with the eyelid closed, providing significant benefit in reducing injury to cornea 4 or sclera 6 over traditional opthhalmodynamometry.

To facilitate the transmission of red and/or IR light into eye 2 and/or the receipt of red and/or IR light by camera 42, a red and/or IR filter 46 can be disposed in the path of the light output by illumination device 40 and/or in the path of light received by camera 42 to filter out light other than red and/or IR light of desirable wavelengths. The use of red and/or IR filter 46, however, is not to be construed as limiting the invention since it is envisioned that illumination device 40 can be configured to output red and/or IR light, camera 42 can be configured to only detect red and/or IR light, and/or controller 26 can be configured to only process images in the red and/or IR spectrum whereupon the use of red and/or IR filter 46 is obviated.

In use, imaging device 20 is held in operative relation to eye 2 by a fixation device 50 which supports imaging device 20 and illumination device 40 so that red and/or IR light output by illumination device 40 can enter eye 2 and camera 42 is positioned with the interior of eye 2, especially CRV 10, in field-of-view 44. A head strap 52 can secure fixation device 50 and, hence, imaging device 20 and illumination device 40 in operative relation to eye 2. The illustration of imaging device 20 including illumination device 40 and camera 42 in a common housing is not to be construed as limiting the invention since it is envisioned that illumination device 40 and camera 42 can be housed separately if desired. Accordingly, the illustration of imaging device 20 in FIG. 1 is not to be construed as limiting the invention.

The use of apparatus 18 to determine ICP will now described.

At any suitable and/or desirable time, the pressure of eye 2 in the absence of any load applied thereto, e.g., by pressure loading device 22, is measured by any suitable and/or desirable means, such as, without limitation, a tonometer. When it is desired to acquire images of the interior of eye 2, imaging device 20 is positioned in operative relation to eye 2 and pressure loading device 22 is placed in contact with the exterior of eye 2.

Pressure loading device 22 can be any useful and/or desirable device that can apply a load to eye 2 while, at the same time, enabling camera 42 to observe structure within eye 2, especially CRV 10. Pressure loading device 22 can be the combination of a suction cup affixed to the exterior of eye 2 and a vacuum source coupled to the suction cup to apply a vacuum (negative pressure) to eye 2 under the control of controller 26 whereupon the internal pressure inside eye 2 increases in response to decreasing the volume enclosed by the exterior of eye 2. Alternatively, pressure loading device 22 can be any suitable device that can be utilized to apply a pressing force (positive pressure) to eye 2 whereupon the internal pressure inside eye 2 increases in response to decreasing the volume enclosed by the exterior of eye 2.

Once pressure loading device 22 is positioned on eye 2 and imaging device 20 is positioned in operative relation to eye 2, controller 26 causes pressure loading device 22 to continuously or step increase the internal pressure within eye 2. Desirably, during the time the internal pressure of eye 2 is being increased, camera 42 acquires a plurality of electronic images of the interior of eye 2, especially CRV 10, in field-of-view 44 of camera 42 under the control of controller 26. As discussed above, camera 42 desirably receives red and/or IR light from the interior of eye 2. Hence, each electronic image acquired by camera 42 is a red and/or IR image. If desired, controller can convert each red and/or IR image into a corresponding image in the visible spectrum and can cause each image in the visible spectrum to be displayed on display 28.

Pressure transducer 24 is configured to monitor the load applied to the exterior of eye 2 by pressure loading device 22, and to convert this load into a corresponding electronic signal for processing by controller 26. While a single pressure transducer 24 is illustrated, it is envisioned that two or more pressure transducers 24 can be utilized to detect the load being applied to the exterior of eye 2. Similarly, two or more pressure loading devices can be utilized to apply a load to the exterior of eye 2. Accordingly, the illustration in FIG. 1 of a single pressure loading device 22 and a single pressure transducer 24 is not to be construed as limiting the invention.

Under the control of controller 26, pressure loading device 22 increases the load, and, hence, the internal pressure of eye 2 until one or more portions of CRV 10 collapse in response to the internal pressure of eye 2 increasing to the point where it overcomes the internal pressure of the blood in CRV 10 whereupon at least a portion of CRV 10 collapses. The collapse of CRV 10 can be determined automatically by controller 26 by comparing a first electronic image acquired by camera 42, when CRV 10 is in its open state, to a second electronic image acquired by camera 42, when CRV 10 is in its collapsed state. More particularly, controller 26 determines when CRV 10 collapses by detecting a reduction in the blood volume residing in at least a portion of CRV 10 in two electronic images acquired by camera 42. For example, controller 26 compares an electronic image of the interior of eye 2 when the internal pressure of eye 2 is lower and CRV is in its open state to an electronic image of the interior of eye 2 wherein the internal pressure of eye 2 is higher and CRV is in its collapsed state utilizing suitable image processing techniques. Controller can determine from these electronic images when CRV 10 collapses.

In response to determining that CRV 10 has collapsed, controller 26 samples the output of pressure transducer 24 thereby acquiring an indication of the load applied to the exterior of eye 2.

Utilizing a calibration curve or algorithm that relates the interior pressure of eye 2 to the load applied to eye 2 by pressure loading device 22, along with the resting interior pressure of eye 2 measured without pressure loading device 22 applied to the exterior of eye 2, controller 26 can electronically estimate the actual interior pressure of eye 2 at the time of CRV 10 collapse. More specifically, the interior pressure of eye 2 measured by way of pressure loading device 22 at the time of CRV 10 collapse, also known as the intraocular pressure (IOP), and the resting interior pressure of eye 2 are summed (added) together by controller 26 to obtain the estimate of the actual interior pressure of eye 2 at the time of CRV 10 collapse, also known as venous outflow pressure (VOP).

The calibration curve or algorithm that relates the interior pressure of eye 2 to the load applied to eye 2 by pressure loading device 22 is based on characteristics of pressure loading device 22. For example, if pressure loading device 22 is a suction cup, for a given vacuum applied to the suction cup, the diameter of the suction cup is related to the load applied to eye 2. For example, for two suction cups of different diameters applying a load to the exterior of eye 2 under the influence of the same level of vacuum, the suction cup having the greater diameter will apply a greater load than the suction cup having a smaller diameter. The calibration curve or algorithm can be determined empirically, theoretically, or some combination of empirically and theoretically.

It has been observed that there is a high degree of correlation between ICP and the VOP when CRV 10 collapses. Thus, in response to detecting the collapse of CRV 10 at a load communicated to controller 26 by pressure transducer 24, controller 26 can determine the VOP and, thus, to a high degree of correlation, the corresponding ICP. The ICP determined by controller 26 can be output on display 28 or any other suitable output means, such as a printer, and/or can be stored for subsequent retrieval and analysis.

Also or alternatively, controller 26 can cause electronic images acquired by camera 42 to be displayed on display 28 for viewing by an attendant. In this regard, where the images acquired by camera 2 are in the red and/or IR spectrum, controller 26 can convert the red and/or IR images into images in the visible spectrum for display on display 28. In response to visually detecting the collapse of CRV 10, the attendant can supply a suitable signal indicative of the collapse of CRV 10 to controller 26 via keyboard 30 and/or mouse 32. In response to receiving this signal, controller 26 can acquire the output of pressure transducer 24 and can estimate therefrom and from the resting interior pressure of eye 2 the ICP of the patient.

The resting interior pressure of eye 2 can be entered into controller 26 via keyboard 30 and/or mouse 32 in a manner known in the art. Also or alternatively, the device utilized to measure the resting interior pressure of eye 2 can be equipped to provide to controller 26 a signal indicative of the resting interior pressure of eye 2 thereby obviating the need for the entry of this data into controller 26 via keyboard 30 and/or mouse 32.

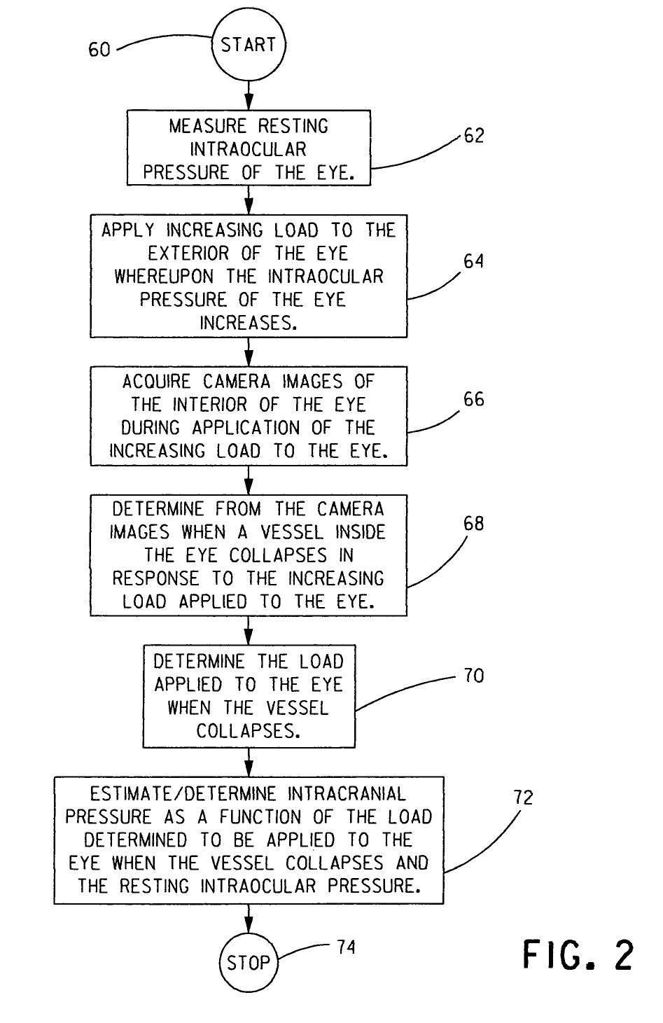

With reference to FIG. 2, a method of estimating or determining ICP advances from start step 60 to step 62 wherein the resting interior pressure of the eye is measured in the absence of a load applied to the exterior of eye 2. The method then advances to step 64 wherein an increasing load is applied to an exterior of the eye whereupon the intraocular or interior pressure of the eye increases. The method then advances to step 66 wherein camera images of the interior of the eye are acquired during application of the increasing load to the eye, desirably in the red and/or IR spectrum.

Thereafter, in step 68, a determination is made from the camera images acquired in step 66 when a vessel inside the eye collapses in response to the increasing load applied to the eye in step 64. This determination can be made by way of a programmed controller or computer which utilizes suitable software techniques, e.g., computer vision and pattern recognition software, to determine when the vessel collapses. Desirably, the vessel being detected for collapse is the central retinal vein (CRV) 10. However, this is not to be construed as limiting the invention since it is envisioned that the collapse of any suitable and/or desirable vessel within the eye can be observed.

The method then advances to step 70 wherein the load applied to the eye when the vessel collapses is determined. Next, in step 72, the intracranial pressure is estimated/determined as a function of the load determined to be applied to the eye in step 70 and the resting intraocular or interior pressure measured in step 62. Thereafter, the method advances to stop step 74 where the method terminates.

The invention has been described with reference to the preferred embodiments. Obvious modifications and alterations will occur to others upon reading and understanding the preceding detailed description. It is intended that the invention be construed as including all such modifications and alterations insofar as they come within the scope of the appended claims or the equivalents thereof.

The invention claimed is:

1. A method of determining intracranial pressure (ICP) of a patient comprising:
(a) observing a vessel in a patient's eye;
(b) causing a pressure inside the eye to increase;
(c) determining when the observed vessel collapses in response to increasing the pressure inside the eye;
(d) estimating the pressure inside the eye on or about the time the vessel collapses; and
(e) estimating the ICP as a function of the estimated pressure inside the eye.

2. The method of claim 1, further including:
determining a resting pressure inside the eye; and
estimating the ICP as a function of the combination of the resting pressure and the estimated pressure inside the eye.

3. The method of claim 1, wherein step (a) includes:
causing light to shine into the patient's eye;
electronically acquiring a plurality of images from the patient's eye when the light is shining therein; and
electronically processing each acquired image.

4. The method of claim 3, wherein at least one of:
the light is in red and/or IR spectrum; or
each image is acquired in the red and/or IR spectrum.

5. The method of claim 3, wherein step (c) includes automatically determining when the vessel collapses from the plurality of electronically processed images.

6. The method of claim 1, wherein the vessel is observed at wavelengths between one of 400-2500 nm, 400-1000 nm, 500-1000 nm, 600-1000 nm and 600-700 nm.

7. The method of claim 1, wherein:
step (a) is accomplished by detecting blood volume in the vessel in the red and/or IR spectrum; and
step (c) is accomplished by detecting a reduction in the blood volume in at least a portion of the vessel in the red and/or IR spectrum.

8. The method of claim 1, wherein the vessel is the central retinal vein of the eye.

9. An apparatus for determining intracranial pressure (ICP) of a patient comprising:
a camera for electronically acquiring a plurality of images of an interior of an eye of the patient;
a pressure loading device for applying a load to an exterior of the eye to increase a pressure inside the eye;
a load detector for electronically determining an amount of the load applied to the exterior of the eye by the pressure loading device; and
a controller for processing the images acquired by the camera to automatically determine when a vessel in the interior of the eye collapses in response to increasing the pressure inside the eye, for acquiring from the load detector the amount of the load applied to the exterior of the eye by the pressure loading device on or about the time the vessel collapses, and for determining the ICP as a function of amount of the load applied to the exterior of the eye on or about the time the vessel collapses.

10. The apparatus of claim 9, further including a light source for shining light into the interior of the eye.

11. The apparatus of claim 10, wherein at least one of:
the light is in the red and/or infrared (IR) spectrum; or
the camera acquires images in the red and/or IR spectrum.

12. The apparatus of claim 9, further including a system for determining a resting pressure inside the eye in the absence of a load applied to the exterior of the eye, wherein the controller determines the ICP as a function of the combination of the resting pressure inside the eye and the amount of the load applied to the exterior of the eye on or about the time the vessel collapses.

13. The apparatus of claim 9, wherein the controller automatically determines when the vessel collapses by comparing two or more of the acquired images and determining from said comparison when a reduction in the amount of blood volume in the vessel occurs.

14. A method of determining intracranial pressure (ICP) of a patient comprising:
(a) acquiring electronic images of a vessel in an interior of a patient's eye;
(b) applying an increasing load to the exterior of the eye, whereupon a pressure inside the eye increases, until the vessel is determined to collapse from the acquired electronic images;
(c) determining the load applied to the exterior of the eye on or about the time the vessel collapses; and
(d) estimating the ICP as a function of the load applied to the exterior of the eye on or about the time the vessel collapses.

15. The method of claim 14, wherein the electronic images are acquired in the red and/or infrared (IR) spectrum.

16. The method of claim 15, further including:
converting the acquired electronic red and/or IR images into corresponding images in the visible spectrum; and
manually determining when the vessel collapses via the images in the visible spectrum.

17. The method of claim 14, further including:
automatically determining when the vessel collapses from the acquired electronic images;
automatically determining the load applied in step (c); and
automatically determining the ICP in step (d).

18. The method of claim 14, further including determining a resting pressure inside the eye in the absence of a load being applied to the exterior of the eye, wherein step (d) includes estimating the ICP as a function of the resting pressure.

19. The method of claim 18, wherein:
step (c) includes estimating an actual load applied to the eye based on the load determined to be applied to the exterior of the eye on or about the time the vessel collapses and based on at least one characteristic of a device used to apply the increasing load to the eye; and
step (d) includes summing the estimated actual load applied to the eye and the resting pressure.

20. The method of claim 19, wherein the means used to apply the increasing load to the eye applies either a negative pressure or a positive pressure to the exterior of the eye.

21. The method of claim 20, wherein:
the means for applying a negative pressure includes a suction cup coupled to a source of vacuum;
one of the characteristics used to estimate the actual load applied to the eye includes the diameter of the suction cup; and
the load determined to be applied to the exterior of the eye on or about the time the vessel collapses is determined from a pressure transducer.
